(19) United States
(12) Patent Application Publication
(10) Pub. No.: US 2023/0136277 A1
(43) Pub. Date: May 4, 2023

(54) WATER-IN-OIL EMULSIONS INCLUDING SURFACTANT, ACRYLIC POLYMER AND ORGANOSILOXANE

(71) Applicant: L'OREAL, PARIS (FR)

(72) Inventors: Geoffrey WHITE, Old Bridge, NJ (US); Aline Aude GUIMONT, SOUTH ORANGE, NJ (US)

(73) Assignee: L'OREAL, PARIS (FR)

(21) Appl. No.: 17/513,988

(22) Filed: Oct. 29, 2021

(51) Int. Cl. A61K 8/06; A61K 8/25; A61K 8/37; A61K 8/81; A61K 8/92; A61K 8/891; A61K 8/91; A61Q 1/10

(52) U.S. Cl. CPC A61K 8/064; A61K 8/25; A61K 8/37; A61K 8/8147; A61K 8/92; A61K 8/891; A61K 8/91; A61Q 1/10; A61K 2800/30; A61K 2800/43; A61K 2800/5424; A61K 2800/594

(57) ABSTRACT

A water-in-oil emulsion composition comprising (a) water, (b) at least one oil, (c) at least one surfactant, (d) at least one amine salt of an acrylic polymer, and (e) at least one organosiloxane film-former that is selected from a silicone resin and a random acrylic/silicone graft copolymer is provided. The composition may be a composition for eyelashes, for example a mascara composition further including at least one colorant.

### FIELD OF THE INVENTION

The present invention relates to water-in-oil (w/o) emulsions (compositions) comprising (1) at least one surfactant (2) at least one amine salt of an acrylic polymer, and (3) at least one organosiloxane film-former. The compositions have beneficial cosmetic properties including good or improved wear, sebum resistance and/or water resistance properties. Significantly, the compositions also have good or improved sebum resistance and removal properties.

### DISCUSSION OF THE BACKGROUND

Mascara compositions are commonly used to enhance the appearance of eyelashes. Conventional mascara compositions generally use waxes to form crystalline network structures to enhance curl, volume, length, thickness, and/or colors to eyelashes. However, conventional mascara compositions including waxes tend to become less resistant to oil and/or sebum, causing smearing, flaking, and/or color transferring after wearing for a certain amount of time.

Mascaras are in particular prepared on the basis of two types of formulations: aqueous mascaras referred to as cream mascaras, in the form of a dispersion of waxes in water, and anhydrous or low-water-content mascaras, referred to as water-resistant mascaras (referred to as “waterproof”), in the form of dispersions of waxes in organic solvents.

The aqueous mascaras mainly contain a surfactant system, for example based on triethanolamine stearate, which makes it possible to obtain a stable dispersion of particles of wax agglomerated in an aqueous phase. This system plays an important part in the obtaining of such a dispersion, in particular at the interface in the interactions between particles of wax.

Anhydrous mascaras tend to have the disadvantages of, among other things, being dry, having poor flexibility and/or having poor consistency. Also, consumers have difficulty removing such mascaras from eyelashes.

WO 2019/122102 is directed to a direct emulsion for use on lips containing at least 10% silicone resin.

WO 2014/046883 is directed to a slurry powder cosmetic composition containing particulates coated with a binder composition, a silicone gel composition, and the evaporation residue of an alcohol based composition and a method for making the composition.

WO 2010/149493 is directed to composition for making up and/or caring for keratin materials, comprising at least one fatty phase, at least one silicone resin and at least one styrene/acrylate copolymer.

WO 2014/143757 is directed to compositions containing a silicone resin emulsion containing 0.5 wt % to 95 wt % of a silicone resin or pressure sensitive adhesive (PSA), 0.1 to 90 wt % of a ethylene oxide/propylene oxide block copolymer, and sufficient amount of water to sum to 100 weight percent.

U.S. Pat. No. 10,441,527 is directed to fluid compositions containing multiple components, the first of which can include a copolymer which is a cross-linked siloxane (e.g. a cross-linked aminosiloxane), a silicone polyether copolymer (e.g. an (AB)n silicone polyether copolymer), and/or a saccharide siloxane copolymer, and the second of which can include organopolysiloxane resin and/or an acrylate copolymer.

FR 2968983 is directed to compositions fluid anhydrous compositions containing at least one organopolysiloxane elastomer powder coated with silicone resin; at least one organopolysiloxane elastomer vehicle in a first oil; at least one wax; at least one organic lacquer; and at least a second oil.

US 2006/0078578 is directed to film forming compositions containing dispersed non-quaternary montmorillonite mineral and a film forming component.

There is a need to develop a cosmetic composition, in particular for making up the eyelashes, making it possible to obtain a good wearing, water- and sebum-resistant and stable and/or easy-to-remove product.

### SUMMARY OF THE INVENTION

The present invention relates to water-in-oil (w/o) emulsions for hair, eyelashes and/or eyebrows comprising at least one surfactant, at least one amine salt of an acrylic polymer preferably without styrene groups, and at least one organosiloxane film-former that is selected from a silicone resin and a random acrylic/silicone graft copolymer. Preferably, the composition is a composition for eyelashes. Most preferably, the composition is a mascara composition further comprising at least one colorant. In certain embodiments, the composition is substantially free of acrylic polymers including styrene groups.

In another aspect, the present invention relates to water-in-oil (w/o) emulsion compositions for eyelashes and/or eyebrows (e.g., a mascara) comprising water, at least one oil, at least one wax in an amount from about 10% to about 25% by weight of the total weight of the composition. It further comprises a low HLB value surfactant comprising a backbone and pendant group(s), wherein (1) the backbone is hydrophobic and one or more pendant groups is/are hydrophilic or (2) the backbone is hydrophilic and one or more pendant groups is/are hydrophobic. It further comprises at least one silicone gum and an amine salt of an acrylic polymer (preferably without styrene groups); an organosiloxane film-former that is selected from a silicone resin and a random acrylic/silicone graft copolymer; and optionally at least one colorant. In certain embodiments, the composition is substantially free of acrylic polymers including styrene groups.

The present invention also relates to methods of treating, caring for and/or making up hair, eyelashes, and/or eyebrows by applying compositions of the present invention to hair, eyelashes, and/or eyebrows in an amount sufficient to treat, care for and/or make up the hair, eyelashes, and/or eyebrows. Preferably, the composition is a composition for eyelashes. Most preferably, the composition is a mascara composition further comprising at least one colorant.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, an item of clothing or the skin, for example. “Smudge resistance” is a type of “transfer resistance”—in the context of mascara compositions, smudges occur when eyelashes contact skin around the eye area and mascara transfers to the contacted skin area. Transfer resistance may be evaluated by any method known in the art for evaluating such. For example, transfer resistance of a composition may be evaluated by a test which may involve application of the composition to human keratin material such as hair, eyelashes or eyebrows followed by rubbing a material, for example, a sheet of paper, against the hair, eyelashes or eyebrows after expiration of a certain amount of time following application, such as 2 minutes after application. Similarly, transfer resistance of a composition may be evaluated by the amount of product transferred from a wearer to any other substrate, such as transfer from the hair, eyelashes or eyebrows of an individual to an item of clothing or skin after the expiration of a certain amount of time following application of the composition to the hair, eyelashes or eyebrows. The amount of composition transferred to the substrate (e.g., collar, skin or paper) may then be evaluated and compared. For example, a composition may be transfer resistant if a majority of the product is left on the wearer's hair, eyelashes or eyebrows. Further, the amount transferred may be compared with that transferred by other compositions, such as commercially available compositions. In a preferred embodiment of the present invention, little or no composition is transferred to the substrate from the hair, eyelashes or eyebrows.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, eyelashes or eyebrows and evaluating the color of the composition after an extended period of time. For example, the color of a composition may be evaluated immediately following application to hair, eyelashes or eyebrows and these characteristics may then be re-evaluated and compared after a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. For example, a composition may be waterproof if a majority of the product is left on the wearer, e.g., eyelashes. In a preferred embodiment of the present invention, little or no composition is transferred from the wearer.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Stability,” as used herein, means that the emulsion does not phase separate at room temperature over a period of at least 2 months.

“Removal properties,” as used herein, refers to removing compositions from hair, eyebrows and/or eyelashes. “Easy removal properties” for a removal process refer to processes which are not as time-consuming and/or which do not substantially damage hair, eyebrows and/or eyelashes. Removal properties can be evaluated, for example, by comparing removability of a composition using a removal solvent (for example, a water-based composition containing soap/surfactant or an oil-based composition) with removability of other compositions (including commercially-available compositions) using the same solvent. For example, compositions being evaluated can be applied to hair, eyebrows and/or eyelashes (real or artificial), and allowed to dry for a designated period of time (for example, 15 minutes). Then, the number of strokes it takes to completely remove the composition from the hair, eyebrows and/or eyelashes using a removal solvent and cotton pad can be counted and compared with other compositions.

The compositions and methods of the present invention can comprise, consist of, or consist essentially of the essential elements and limitations of the invention described herein, as well as any additional or optional ingredients, components, or limitations described herein or otherwise useful. For example, the surfactant component of the emulsion composition can “consist essentially of” or “consist of” low HLB surfactant(s) comprising a backbone and pendant group(s), wherein (1) the backbone is hydrophobic and one or more pendant groups is/are hydrophilic, (2) the backbone is hydrophilic and one or more pendant groups is/are hydrophobic, or (3) both (1) and (2) are present in the surfactant component. Also for example, the film-forming component of the emulsion composition can “consist essentially of” or “consist of” an amine salt of an acrylic polymer preferably without styrene groups; and an organosiloxane film-former that is selected from a silicone resin and a random acrylic/silicone graft copolymer and siloxysilicate resin(s).

The “surfactant component” of the emulsion composition of the present invention may comprise at least one low HLB surfactant comprising a backbone and pendant group(s), wherein (1) the backbone is hydrophobic and one or more pendant groups is/are hydrophilic or (2) the backbone is hydrophilic and one or more pendant groups is/are hydrophobic. It may optionally further contain one or more additional surfactants. Preferably, the surfactant component contains a majority (greater than 50% by weight) of low HLB surfactant comprising a backbone and pendant group(s), wherein (1) the backbone is hydrophobic and one or more pendant groups is/are hydrophilic or (2) the backbone is hydrophilic and one or more pendant groups is/are hydrophobic, preferably greater than 60% by weight, preferably greater than 70% by weight, preferably greater than 80% by weight, preferably greater than 90% by weight, and may contain up to 100% by weight of these surfactants, all percentages being based on total weight of the low HLB surfactant(s) with respect to the total weight of the surfactant component. All ranges and subranges within the percentages set forth above are included herein such as, for example, 10%-100% by weight, 25%-90% by weight, 50%-85% by weight, etc.

The “surfactant component” of the emulsion composition of the present invention preferably comprises from 1% to 15% by weight with respect to the total weight of the composition, preferably 2% to 10% by weight with respect to the total weight of the composition, and preferably from 3% to 8% by weight with respect to the total weight of the composition, including all ranges and subranges therebetween.

The “film-forming component” of the emulsion composition of the present invention comprises an amine salt of an acrylic polymer preferably without styrene groups. The film-forming component also comprises an organosiloxane film-former that is selected from a silicone resin and a random acrylic/silicone graft copolymer.

The “film-forming component” may optionally further contain one or more additional film-forming agents. Preferably, the film-forming component contains a majority of an amine salt of an acrylic polymer and the aforementioned organosiloxane film-former (greater than 50% by weight, combined of both of these film-forming polymers), preferably greater than 60% by weight, preferably greater than 70% by weight, preferably greater than 80% by weight, preferably greater than 90% by weight, and may contain up to 100% by weight of these film-forming agents, all percentages being based on total weight of the two types of film-forming agents with respect to the total weight of the film-forming component. All ranges and subranges within the percentages set forth above are included herein such as, for example, 10%-100% by weight, 25%-90% by weight, 50%-85% by weight, etc.

The “film-forming component” of the emulsion composition of the present invention preferably comprises from 1% to 15% by weight with respect to the total weight of the composition, preferably 2% to 15% by weight with respect to the total weight of the composition, and preferably from 6% to 14% by weight with respect to the total weight of the composition, including all ranges and subranges therebetween.

Amine Salt of an Acrylic Polymer

In accordance with the present invention, water-in-oil emulsion compositions comprising (1) at least one amine salt of an acrylic polymer. By “amine salt of an acrylic polymer,” it is meant a carboxyfunctional (co)polymer of acrylic monomers that is at least partially neutralized with an amine. The amine may be, for example, ammonium (hydroxide), or an alkyl amine, such as triethyl amine, tributyl amine, n,n-diethyl aniline and n,n-diethyl methyl amine The polymer is generally prepared from ethylenically unsaturated monomers, e.g., those derived from acrylic acid, methacrylic acid and/or other acrylate monomers.

According to certain embodiments the amine salt of an acrylic polymer is a solution polymer, i.e., is prepared via solution polymerization, e.g., by dissolving monomer in solvent and adding initiator and other polymerization assisting components.

According to other embodiments, the amine salt of an acrylic polymer is an emulsion polymer (e.g., latex) rather than a solution polymer. A “latex” is generally obtained by suspension or emulsion polymerization or copolymerization of monomers according to processes that are well known to those of ordinary skill in the art. Such monomers may be chosen in particular from acrylic monomers such as acrylic or methacrylic acid, and optionally other monomers such as maleic acid, crotonic acid or itaconic acid or esters or amides thereof.

In accordance with preferred embodiments of the present invention, the water-in-oil emulsion compositions comprise at least one amine salt of an acrylic polymer is free of styrene groups. The amine salt of an acrylic polymer that is free of styrene groups may, in certain embodiments be a solution polymer.

According to certain embodiments, the amine salt of an acrylic polymer may comprises a polymer chosen from copolymers resulting from the polymerization of at least one C₁-C₁₈ alkyl (meth)acrylate monomer, preferably C₁-C₁₂ alkyl (meth)acrylate, and preferably C₁-C₁₀ alkyl (meth)acrylate. The C₁-C₁₈ alkyl (meth)acrylate monomer is preferably chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate. While in certain embodiments, the monomers used are all free of styrene groups, in certain embodiments styrene-containing monomers are included. As styrene monomer that may be used in the invention, examples that may be mentioned include styrene and α-methylstyrene. Separately, in certain embodiments, the entire emulsion composition is substantially free of acrylic polymers that include styrene groups.

According to certain embodiments, the amine salt of an acrylic polymer has a high molecular weight. As used herein, “high molecular weight” means more than 100,000 Da weight average molecular weight, preferably more than 120,000 Da, preferably more than 135,000 Da, and preferably more than 150,000 Da, including all ranges and subranges therebetween such as, for example, 100,000 Da-500,000 Da, 120,000 Da to 600,000 Da, 150,000 Da to 200,000 Da, etc.

The amine salt of an acrylic polymer may have a glass transition temperature (Tg) ranging from about −15° C. to about 90° C., such as from about 0° C. to about 50° C., such as from about 5° C. to about 25° C., such as from about 10° C. to about 20° C., including all ranges and subranges therebetween.

According to preferred embodiments, the amine salt of an acrylic polymer is selected from an ammonium acrylates copolymer, a styrene/acrylates/ammonium methacrylate copolymer or combinations thereof. According to certain other embodiments the amine salt of an acrylic polymer is an ammonium acrylates copolymer.

A particular preferred amine salt of an acrylic polymer is ammonium acrylates copolymer, a commercial example of which is SYNTRAN KL-219 from Interpolymer Corporation.

Other notable copolymers include a styrene/acrylates/ammonium methacrylate copolymer or a styrene acrylic copolymer, commercial examples of which include SYNTRAN 5620 and SYNTRAN 5760 (with or without paraben), by Interpolymer Corporation; JONCRYL 77, by BASF Performance Chemicals; and RHOPLEX P376, by Dow Chemical Company.

Preferably, the amine salt of an acrylic polymer is present in the compositions of the present invention in an amount ranging from about 2% to about 15%, more preferably from about 3% to about 12%, and preferably from about 3% to about 8%, by weight, based on the total weight of the composition, including all ranges and subranges in between.

According to preferred embodiments, the amine salt of an acrylic polymer is present in an amount ranging from about 2% to about 6%, preferably from about 2.25% to about 5.5%, and preferably from about 2.5% to about 5%, by weight, based on the total weight of the composition, including all ranges and subranges in between.

Silicone Gum

According to preferred embodiments of the present invention, compositions comprising at least one silicone gum resin are provided. The silicone gum can, for example, correspond to the formula:

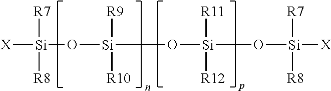

in which:

R₇, R₈, R₁₁ and R₁₂ are identical or different, and each is chosen from alkyl radicals comprising from 1 to 6 carbon atoms,

R₉ and R₁₀ are identical or different, and each is chosen from alkyl radicals comprising from 1 to 6 carbon atoms and aryl radicals,

X is chosen from alkyl radicals comprising from 1 to 6 carbon atoms, a hydroxyl radical and a vinyl radical,

n and p are preferably chosen so as to give the silicone gum a viscosity of from 25,000 cSt to 20,000,000 cSt, preferably from 30,000 cSt to 5,000,000 cSt, preferably from 35,000 cSt to 1,000,000 cSt, preferably from 40,000 cSt to 750,000 cSt, preferably from 50,000 cSt to 500,000 cSt and preferably from 60,000 cSt to 300,000 cSt, including all ranges and subranges therebetween. A particularly preferred range is from 60,000 cSt to 300,000 cSt (corresponding to a molecular weight of 60,000-110,000), with mixtures of two silicone gums within this range being preferred, the weight ratio of the two silicone gums preferably ranging from about 2:1 to 1:2, with a weight ratio of the two silicone gums of about 1:1 being preferred. So, for example, a silicone gum having a viscosity of 60,000 cSt and a silicone gum having a viscosity of 300,000 cSt at a weight ratio of about 1:1 is preferred. Of course, any two silicone gums falling within the identified viscosity ranges and ratios can be used.

In general, n and p can each take values ranging from 0 to 10,000, such as from 0 to 5,000.

Among the silicone gums which can be used according to the invention, mention may be made of those for which:

the substituents R₇ to R₁₂ and X represent a methyl group, p=0 and n=2 700, such as the product sold or made under the name SE30 by the company General Electric,

the substituents R₇ to R₁₂ and X represent a methyl group, p=0 and n=2 300, such as the product sold or made under the name AK 500 000 by the company Wacker,

the substituents R₇ to R₁₂ represent a methyl group, the substituent X represents a hydroxyl group, p=0 and n=2 700, as a 13% solution in cyclopentasiloxane, such as the product sold or made under the name Q2-1401 by the company Dow Corning,

the substituents R₇ to R₁₂ represent a methyl group, the substituent X represents a hydroxyl group, p=0 and n=2 700, as a 13% solution in polydimethylsiloxane, such as the product sold or made under the name Q2-1403 by the company Dow Corning, and

the substituents R₇, R₈, R₁₁, R₁₂ and X represent a methyl group and the substituents R₉ and R₁₀ represent an aryl group, such that the molecular weight of the gum is about 600 000, for instance the product sold or made under the name 761 by Rhodia Chimie.

In preferred embodiments, the silicone gum correspond to the following formula:

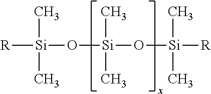

In this formula the terminal Si's can also be other than methyl and may be represented with substitutions on the repeating Si such that the R group is an alkyl of 1 to 6 carbon atoms, which may be linear, branched and/or functionalized selected from methyl, ethyl, propyl, isopropyl, butyl, isobutyl, t-butyl, amyl, hexyl, vinyl, allyl, cyclohexyl, phenyl, and mixtures thereof. The silicone gums employed in the present invention may be terminated by triorganosilyl groups of the formula R′₃ where R′ is a radical of monovalent hydrocarbons containing from 1 to 6 carbon atoms, hydroxyl groups, alkoxyl groups and mixtures thereof.

The at least one silicone gum is preferably present in the compositions of the present invention an amount which is equal to or less than the amount of siloxysilicate resin present, preferably in an amount of less than 15% by weight, preferably less than 10% by weight, preferably less than 5% by weight, preferably less than 3% by weight, including all ranges and subranges therebetween such as, for example, 1% to 15% by weight, 3% to 12% by weight, 1% to 5% by weight, 1% to 3% by weight, 2% to 8% by weight, etc., all weights being based on the weight of the composition as a whole.

Polyorganosiloxane Film Former

According to the present invention, compositions comprising an polyorganosiloxane film-former are provided. The polyorganosiloxane film-former (also referred to herein as organosiloxane film former) is selected from a silicone resin, a random acrylic/silicone graft copolymer, and combinations thereof.

Suitable silicone resins include siloxysilicate resins. Siloxysilicate resins generally have the formula;

[(R)₃SiO]ₓ(SiO_{4/2})_{y}

Where R is an alkyl group preferably comprising 1 to 8 carbon atoms. One non-limiting example of a siloxysilicate in accordance with the present invention is trimethylsiloxysilicate, which may be represented by the following formula:

[(CH₃)₃SiO]ₓ(SiO_{4/2})_{y}.

In the above formulas, x and y preferably range between numbers such as, 10 and 150, 25 and 125, 35 and 100, and 50 to 80, for example.

Preferred siloxysilicate resins are commercially available from General Electric, Dow Corning, Wacker, Milliken, Siltech, Grant Industries, Momentive and Shin-Etsu Silicones under the tradename Resin MQ®.

Other suitable silicone resins include silsesquioxane resins. Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film-forming resin is made up predominantly of tri-functional units (or T units), it is generally called a silsesquioxane resin, which is described, for example in US 2006/0292096, herein incorporated by reference in its entirety.

Examples of silsesquioxane resins that may be used in the present invention are alkyl silsesquioxane resins that are silsesquioxane homopolymers and/or copolymers having an average siloxane unit of the general formula R¹ₙ SiO_{(4-n)/2}, wherein each R₁ is a propyl group, wherein more than 80 mole % of R₁ represent a C₃-C₁₀ alkyl group, n is a value of from 1.0 to 1.4, and more than 60 mole % of the copolymer includes R¹ SiO_{3/2} units. As each R₁ is a propyl group these polymers are called polypropylsilsesquioxane resins or “t-propyl” silsesquioxane resins. These resins and methods of making them are described, for example in U.S. Pat. No. 8,586,013, 2012/0301415, 2007/0093619, and 2006/0292096, all of which are herein incorporated by reference in their entirety.

A non-limiting example of a polypropylsilsesquioxane resin suitable for use in the present invention is commercially available from Dow Corning as Dow Corning 670 Fluid or Dow Corning 680 Fluid. These Dow Corning resins have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer includes RSiO_{3/2} units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about −5° C. to about 5° C.

In certain embodiments, the polyorganosiloxane film-former is a random acrylic/silicone graft copolymer. Suitable random acrylic/silicone graft copolymers include polymers comprising a siloxane group and a hydrocarbon group. For example, suitable polymers include polymers comprising a hydrocarbon backbone such as, for example, a backbone chosen from vinyl polymers, methacrylic polymers, and/or acrylic polymers and at least one chain chosen from pendant siloxane groups, and polymers comprising a backbone of siloxane groups and at least one pendant hydrocarbon chain such as, for example, a pendant vinyl, methacrylic and/or acrylic groups.

The random acrylic/silicone graft copolymer may be selected from polymers derived from non-polar silicone copolymers comprising repeating units of at least one polar (meth)acrylate unit and vinyl copolymers grafted with at least one non-polar silicone chain. In certain embodiments, the random acrylic/silicone graft copolymer has an acrylate backbone, randomly grafted with linear or dendritic silicone. Suitable examples include ACRYLATES/DIMETHICONE COPOLYMERS such KP-550 available from Shin Etsu.

In certain other embodiments, the random acrylic/silicone graft copolymer has a silicone backbone, grafted with acrylate.

The at least one polyorganosiloxane film-former is preferably present in the compositions of the present invention in an amount of less than 15% by weight, preferably less than 10% by weight, including all ranges and subranges therebetween such as, for example, 1% to 15% by weight, 3% to 12% by weight, 1% to 10% by weight, 3% to 8% by weight, etc., all weights being based on the weight of the composition as a whole.

Surprisingly, it has been found that water-in-oil (w/o) emulsion compositions comprising at least one amine salt of an acrylic polymer, an organosiloxane film-former that is selected from a silicone resin and a random acrylic/silicone graft copolymer have both good removal properties as well as resistance to sebum resistance. Surprisingly, using different water dispersible polymers even different water-dispersible acrylic polymers and/or using different organosilicone film formers (e.g., “silicone acrylate” polymers that are block copolymers formed from block copolymeriation of silioxane-containing monomers and acrylate monomers rather than random acrylic/silicone graft copolymers) does not give rise to this combination of properties.

According to particularly preferred embodiments, the combined amount of amine salt of an acrylic polymer and the aforementioned organosiloxane film-former present ranges from about 5% to 25% by weight, preferably from about 7.5% to about 20%, preferably about 10% to about 15% by weight based on the total weight of the composition, including all ranges and subranges within these ranges. Also, the weight ratio of polyorganosiloxane film-former to amine salt of acrylic polymer preferably ranges from 1:1 to about 5:1, preferably from about 2:1 to about 4:1.

Surfactant

According to the present invention, compositions comprising at least one surfactant are provided. Preferably, the at least one surfactant is a low HLB surfactant. “HLB” refers to the “hydrophilic-lipophilic balance” associated with emulsifiers. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “low HLB surfactant” means a surfactant having an HLB value of less than or equal to 6, preferably less than 5, and preferably less than 4. Preferably, the HLB value is 3 or greater.

The surfactant, in particular a low HLB value surfactant, is preferably present as all or part of a “surfactant component” in the compositions of the present invention. According to preferred embodiments of the present invention, the surfactant component has an HLB value of less than or equal to 6, preferably less than 5, and preferably less than 4. Preferably, the HLB value is 3 or greater.

According to preferred embodiments, the surfactant, in particular a low HLB surfactant, is nonionic.

According to preferred embodiments, the surfactant, in particular a low HLB surfactant, comprises a backbone and pendant group(s), wherein (1) the backbone is hydrophobic and one or more pendant groups is/are hydrophilic, or (2) the backbone is hydrophilic and one or more pendant groups is/are hydrophobic. The backbone can be silicone-based or hydrocarbon based. By “backbone,” it is meant that the surfactant comprises a main chain. By “pendant” group(s), it is meant that one or more groups is attached to the backbone or main chain of the surfactant. The pendant group(s) can be attached at any location along the backbone or main chain, for example at one or both terminal ends of the chain, at location(s) not at a terminal end of the chain, or both. According to preferred embodiments, the pendant group(s) include one or more ester group(s) attached to the backbone or main chain.

An especially preferred backbone is a hydrophobic, silicone-based backbone such as dimethicone. For such a backbone, a preferred pendant group is a hydrophilic group such as one including a plurality of ether groups such as a polyethylene glycol (PEG) polymer.

Another preferred backbone is a hydrophilic, hydrocarbon-based backbone such as a polyethylene glycol (PEG) polymer or a polyglyceryl polymer. For such a backbone, a preferred pendant group is an ester based on a hydrophobic group such as, for example, a C8-C24 fatty compound, preferably a C12-C20 fatty compound, and preferably a C16-C18 fatty compound such as, for example, stearate, isostearate, laurate, etc.

According to preferred embodiments, compositions of the present invention comprise at least one surfactant comprising a silicone-based backbone and at least one surfactant comprising a hydrocarbon-based backbone.

According to preferred embodiments, the surfactant component comprises surfactant(s), in particular low HLB surfactant(s), comprising a backbone and pendant group(s), wherein (1) the backbone is hydrophobic and one or more pendant groups is/are hydrophilic, (2) the backbone is hydrophilic and one or more pendant groups is/are hydrophobic, or (3) both (1) and (2) are present in the surfactant component.

The surfactant, in particular a low HLB surfactant, of the present invention can be a silicone surfactant, preferably comprising dimethicone (for example, PEG-10 dimethicone or lauryl PEG-9 polydimethylsiloxyethyl dimethicone), or a non-silicone surfactant, free of dimethicone (for example, PEG-30 dihydroxystearate). The surfactant, in particular a low HLB surfactant, of the present invention can have alkoxylation (for example, ethoxylation or propoxylation) through groups such as, for example, (C₂H₄O)—/polyethylene glycol groups (PEG), (C₃H₆O)—/polypropylene glycol groups (PPG), etc. Suitable emulsifiers include alkoxylated compounds, partial glycerides of alkoxylated compounds, polyglycerolated compounds, and mixtures thereof, etc.

According to preferred embodiments, the surfactant, in particular a low HLB surfactant, can be a silicone surfactant which is an oxyalkylenated organosiloxane. Preferably, the organosiloxane emulsifier is linear.

For example, a low HLB surfactant can have the general formula:

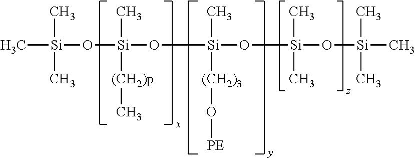

wherein p is 0-40 (the range including all numbers between and subranges such as 2, 3, 4, 13, 14, 15, 16, 17, 18, etc.), and PE is (—C₂H₄O)ₐ-(—C₃H₆O)_{b}H wherein a is 0-25, b is 0-25 with the proviso that both a and b cannot be 0 simultaneously, x, y, and z are each independently ranging from 0 to 1 million with the proviso that x and y cannot be 0 simultaneously. In some cases, x, y, z, a, and b are such that the molecular weight of the polymer ranges from about 5,000 to about 500,000, from about 10,000 to 100,000, or is about 50,000. In some instances, p is such that the long chain alkyl is cetyl or lauryl, and the compound is called, generically, cetyl dimethicone copolyol or lauryl dimethicone copolyol respectively. In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or ether, such as methyl ether, ethyl ether, propyl ether, butyl ether, and the like.

Further the surfactant, in particular a low HLB surfactant, can have the general formula:

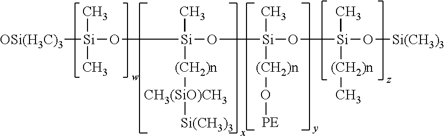

wherein each n is independently 0-100 with the proviso that there must be at least one PE radical. In some instances, where each n independently ranges from about 2 to 30, and PE (—C₂H₄O)ₐ-(—C₃H₆O)_{b}H wherein a is 0-25, b is 0-25 with the proviso that both a and b cannot simultaneously be 0; and wherein w, x, y, and z are each independently 0 to 1,000,000 with the proviso that there is at least one PE. In some embodiments the organosiloxane emulsifier is lauryl PEG-9 Polydimethylsiloxyethyl Dimethicone. Oxyalkylenated organosiloxane emulsifiers disclosed in U.S. Pat. No. 9,095,543 are useful in the instant compositions. U.S. Pat. No. 9,095,543 is incorporated herein by reference in its entirety.

Further examples of organosiloxane emulsifiers include but are not limited to those having C.T.F.A. names Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bis(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-10 Phosphate; Dimethicone PEG/PPG-7/4 Phosphate; Dimethicone PEG/PPG-12/4 Phosphate; Dimethicone PEG-7 Undecylenate; Lauryl Dimethicone PEG-10 Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone; PEG-11 Methyl Ether Dimethicone; PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEG/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; PEG/PPG-18 dimethicone, PEG/PPG-18/18 dimethicone; lauryl PEG/PPG-18/18 methicone; cetyl PEG/PPG-14/14 dimethicone; bis-cetyl PEG/PPG-14/14 dimethicone; cetyl PEG/PPG-10/1 dimethicone; PEG-11 methyl ether dimethicone; PEG/PPG-20/22 butyl ether dimethicone; PEG-9 dimethicone; PEG-3 dimethicone; PEG-9 methyl ether dimethicone; PEG-10 dimethicone; lauryl PEG-9 polydimethylsiloxyethyl dimethicone. or mixtures thereof.

Examples of commercial linear organosiloxane emulsifiers are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Particularly preferred low HLB surfactants are as shown in Table 1:

TABLE 1

| Surfactants | HLB |
|---|---|
| POLYGLYCERYL-2 DIISOSTEARATE | 5.5 |
| GLYCOL DISTEARATE | 5 to 6 |
| GLYCERYL STEARATE | 5.8 |
| PEG-30 DIPOLYHYDROXYSTEARATE | 5.5 |
| PEG-10 DIMETHICONE | 4.5 |
| LAURYL PEG-9 POLYDIMETHYLSILOXYETHYL | 3 |
| DIMETHICONE | |
| PEG-9 POLYDIMETHYLSILOXYETHYL DIMETHICONE | 4 |
| PEG-6 DIISOSTEARATE | 5 |

The at least one surfactant, in particular a low HLB surfactant, is preferably present in the compositions of the present invention in an amount of about 1% to about 15%, preferably from about 1.5% to about 10%, and preferably from about 2% to about 8% by weight with respect to the total weight of the composition, including all ranges and subranges therebetween.

Water

According to the present invention, compositions comprising water are provided. Compositions of the present invention comprise sufficient water to form a water-in-oil emulsion, preferably from about 5% to about 80% water, more preferably from about 10% to about 50% water, and more preferably from about 15% to about 30% water by weight with respect to the total weight of the composition, including all ranges and subranges therebetween.

Oil

According to the present invention, compositions comprising at least one oil are provided. Compositions of the present invention comprise sufficient oil to form a water-in-oil emulsion, preferably from about 5% to about 80% oil, more preferably from about 10% to about 50% oil, and more preferably from about 15% to about 30% oil by weight with respect to the total weight of the composition, including all ranges and subranges therebetween.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 3 below.

TABLE 3

| | Flash Point |
|---|---|
| Compound | (° C.) |
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearly alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

Compositions of the present invention can optionally further comprise any additive usually used in the field(s) under consideration. For example, dispersants, antioxidants, sunscreens, preserving agents, fragrances, fibers, fillers, neutralizing agents, cosmetic and dermatological active agents such as, for example, emollients, moisturizers, vitamins, essential fatty acids, additional surfactants, silicone elastomers, pasty compounds, viscosity increasing agents, for example, liposoluble/lipodispersible polymers, additional film forming agents, colorants, and mixtures thereof can be added. A non-exhaustive listing of such ingredients can be found in U.S. patent application publication nos. 2004/0170586 and 2009/0142289, the entire contents of which are hereby incorporated by reference. Further examples of suitable additional components can be found in the other references which have been incorporated by reference in this application.

According to preferred embodiments of the present invention, compositions further comprising at least one wax are provided.

“Wax” means a lipophilic compound, solid at ambient temperature (25° C.), with a reversible solid/liquid change of state, having a melting point greater than or equal to 30° C.

The waxes used in accordance with the present invention can be any wax and is not particularly limited. The waxes can be hydrocarbon, fluorinated and/or silicone, and be of plant, mineral, animal and/or synthetic origin.

Suitable examples of waxes include, but are not limited to, Carnauba wax, candelilla wax, BIS-PEG-12 DIMETHICONE CANDELILLATE wax such as for example the Siliconyl Candelilla Wax marketed by the company KOSTER KEUNEN, hydrogenated Jojoba wax such as for example that marketed by the company DESERT WHALE, hydrogenated palm oil such as that marketed by the company SiO, rice bran wax, Sumac wax, ceresin waxes, laurel wax, Chinese insect wax, Shellac wax, hydrogenated olive oil such as Waxolive from the company SOLIANCE, waxes obtained by hydrogenation of olive oil esterified with C12 to C18 chain fatty alcohols such as those sold by the company SOPHIM under the brand names Phytowax Olive 12L44, 14L48, 16L55 and 18L57, waxes obtained by hydrogenation of castor oil esterified with cetyl or behenyl alcohol such as for example those which are sold under the names Phytowax Ricin 16 L 64 and Phytowax Ricin 22 L 73 by the company SOPHIM, hydrogenated Cameline wax, Ouricury wax, Montan wax, ozokerite waxes such as for example Wax SP 1020 P marketed by the company Strahl & Pitsch, microcrystalline waxes such as for example that sold under the brand name Microwax HW by the company PARAMELT, triglycerides of lauric, palmitic, cetylic and stearic acids (INCI name: hydrogenated coco glycerides) such as for example that sold under the brand name Softisan 100 by the company SASOL, polymethylene waxes such as for example that sold under the brand name Cirebelle 303 by the company SASOL, polyethylene waxes such as for example those sold under the brand names Performalene 400 polyethylene, Performalene 655 polyethylene and Performalene 500-L polyethylene by the company New Phase Technologies, alcohol-polyethylene waxes such as for example that marketed under the name Performacol 425 Alcohol by the company BARECO, the 95/5 ethylene/acrylic acid copolymer sold under the brand name AC 540 wax by the company Honeywell, hydroxyoctacosanyl hydroxy-stearate such as for example that sold under the brand name Elfacos C 26 by the company AKZO, octacosanyl stearate such as for example that marketed under the name Kester Wax K 82H by the company KOSTER KEUNEN, bis-PEG12 dimethicone beeswax such as also marketed by KOSTER KEUNEN, stearyl stearate such as for example that marketed under the name Liponate SS by the company LIPO CHEMICALS, pentaerythritol distearate such as for example that marketed under the name Cutina PES by the company COGNIS, the mixture of dibehenyl adipate, dioctadecyl adipate and di-eicosanyl adipate (INCI name C18-C22 dialkyl adipate), the mixture of dilauryl adipate and ditetradecyl adipate (INCI name: C12-C14 dialkyl adipate), the mixture of dioctadecyl sebacate, didocosyl sebacate and dieicosyl sebacate (INCI name: C18-C22 dialkyl sebacate) and the mixture of dioctadecyl octadecanedioate, didocosyl octanedioate and dieicosyl octanedioate (INCI name: C18-C22 dialkyl octanedioate) such as for example those marketed by the company COGNIS, pentaerythrityl tetrastearate such as for example Liponate PS-4 from the company Lipo Chemicals, tetracontanyl stearate such as for example Kester Wax K76H from the company KOSTER KEUNEN, stearyl benzoate such as for example Finsolv 116 from the company FINETEX, behenyl fumarate such as for example Marrix 222 from the company AKZO BERNEL, di-(trimethylol-1,1,1-propane) tetrastearate such as for example that which is offered under the name “HEST 2T-4S” by the company HETERENE, didotriacontanyl distearate such as for example Kester Wax K82D from the company KOSTER KEUNEN, polyethylene glycol montanate with 4 ethylene oxide units (PEG-4) such as for example that which is sold under the brand name Clariant Licowax KST1, hexanediol disalicylate such as for example Betawax RX-13750 marketed by the company CP Hall, dipentaerythritol hexastearate such as for example that which is sold under the brand name Hest 2P-6S by the company HETERENE, ditrimethylolpropane tetrabehenate such as for example that which is sold under the brand name Hest 2T-4B by the company HETERENE, Jojoba esters such as for example that which is sold under the brand name Floraester HIP by the company FLORATECH, mixtures of linear (C20-40) carboxylic acid/saturated hydrocarbons (INCI name: C20-40 acid polyethylene) such as for example Performacid 350 acid from the company NEW PHASE TECHNOLOGIES, synthetic wax of the Fischer-Tropsch type such as that marketed under the name Rosswax 100 by the company ROSS, cetyl alcohol, stearyl alcohol, behenyl alcohol, dioctadecyl carbonate such as for example Cutina KE 3737, saccharose polybehenate such as for example Cortiderm B from the company CRODA, and mixtures thereof, can in particular be cited. Such waxes are also described in U.S. patent application publication no. 2009/0142289, the entire contents of which is hereby incorporated by reference.

Waxes of plant origin such as carnauba wax, candelilla wax, hydrogenated jojoba wax, sumac wax, waxes obtained by hydrogenation of olive oil esterified with C12 to C18 chain fatty alcohols sold by the company SOPHIM in the Phytowax range (12L44, 14L48, 16L55 and 18L57), rice bran wax, cetyl, stearyl and behenyl alcohols, laurel wax and Ouricury wax are preferably used.

Examples of suitable silicone waxes include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

If present in the composition, the wax(es) are preferably present in a quantity of at least 5% by weight, preferably from about 5 to about 35% by weight, preferably from about 7.5 to about 30% by weight, and preferably from about 10 to about 25% by weight relative to the total weight of the composition, including all ranges and subranges therebetween. According to certain embodiments, the composition comprises at least 1% by weight of one or more waxes having a melting point of greater than 65° C. Examples of such waxes include candelilla wax, carnauba wax, pegylated beeswax such as bis-peg-12 dimethicone beeswax, and combinations thereof.

According to preferred embodiments, the compositions of the present invention comprise at least one additional film forming agent. According to preferred embodiments, the compositions of the present invention comprise, as additional film forming agent, either instead of or in addition to the dispersion of film forming particles in aqueous phase discussed above, one or more film-forming agents suitable for use in compositions for application to eyebrows and/or eyelashes. Such film-forming agents can be, for example, water-soluble or liposoluble. Acceptable film-forming are known in the art and include, but are not limited to, those disclosed in U.S. patent application publication no. 2004/0170586, the entire contents of which is hereby incorporated by reference.

Specific examples of film-forming agents include, but are not limited to, proteins, such as proteins of plant origin, such as, for example, wheat or soya proteins; or proteins of animal origin, such as keratins, for example keratin hydrolysates and sulfonic keratins; cellulose polymers, such as, for example, hydroxyethylcellulose, hydroxypropylcellulose, methylcellulose or ethylhydroxyethylcellulose; gums arabic, guar gum, xanthan derivatives or karaya gum; alginates and carrageenans; glycoaminoglycans, hyaluronic acid and its derivatives; shellac resin, gum sandarac, dammars, elemis or copals; muccopolysaccharides, such as chondroitin sulfates, and mixtures thereof.

Specific examples of suitable polymers further include, but are not limited to, polyalkylenes, polyvinylpyrrolidone (PVP) or vinylpyrrolidone (VP) homopolymers or copolymers, copolymers of a C₂ to C₃₀, such as C₃ to C₂₂ alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer, and mixtures thereof.

According to preferred embodiments, the compositions of the present invention comprise at least one polyorganosiloxane copolymer. The polyorganosiloxane copolymer useful herein is preferably a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane copolymers are disclosed, for example in U.S. Pat. No. 8,945,525, the disclosure of which is hereby incorporated by reference in its entirety.

Additional polyorganosiloxane copolymers which may be used in the long-wear lip compositions of the present invention include those described in documents U.S. Pat. Nos. 5,874,069, 5,919,441, 6,051,216, and 5,981,680, the entire contents of which are hereby incorporated by reference in their entirety.

A preferred polyorganosiloxane copolymer for use in the present invention contain at least one moiety chosen from formula (III):

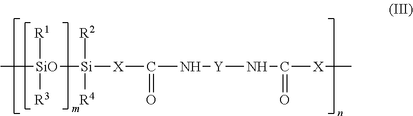

and formula (IV)

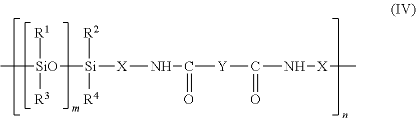

in which:

(a) R1, R2, R3 and R4 are the same or different and may be selected from the group consisting of methyl, ethyl, propyl, isopropyl, a siloxane chain, and phenyl;

(b) X is a linear or branched chain alkylene having 1-30 carbons;

(c) Y is selected from the group consisting of linear or branched chain alkylenes having 1-40 carbons;

(d) m is a number between 1 and 700;

(e) n is a number between 1 and 500.

Particularly preferred polyorganosiloxane copolymers useful herein are commercially available from Dow Corning under the tradenames DC 8178® and DC 8179®, which are known under the INCI denomination of Nylon-611/Dimethicone Copolymer.

If present, the polyorganosiloxane copolymer is preferably present in the compositions of the present invention in an amount ranging from about 1% to about 35% by weight, preferably from about 5% to about 25% by weight, and preferably from about 7% to about 15% by weight, including all ranges and subranges therebetween, all weights being based on the weight of the composition as a whole.

Suitable fillers and/or viscosity increasing agents include silicate clays such as, for example, silicate clays containing at least one cation which may be chosen from calcium, magnesium, aluminium, sodium, potassium, and lithium cations, and mixtures thereof. Non-limiting examples of such products include smectite clays such as montmorillonites, hectorites, bentonites, beidellites, saponites, vermiculites, stevensite, and chlorites. Preferred clays are synthetic silicate clays, most preferably lithium magnesium sodium silicate, commercially available from Rockwood under the tradename Laponite®. Other preferred examples of silicate clays which may be used in the present invention are chosen from lithium magnesium silicate, aluminum calcium sodium silicate, calcium magnesium silicate, sodium magnesium silicate, calcium aluminum borosilicate, magnesium aluminum silicate, sodium potassium aluminum silicate, and sodium silver aluminum silicate. According to certain embodiments the (silicate) clay is hydrophobic, such as rendered by a hydrophobic coating, e,g, disteardimonium hectorite or stearalkonium hectorite.

If present, the filler and/or viscosity increasing agent is preferably present in the compositions of the present invention in an amount of from 0.1% to 10% by weight, preferably in an amount of from 0.5% to 7%, preferably in an amount of from 1% to 5% by weight, based on the total weight of the composition.

According to preferred embodiments of the present invention, the compositions can further comprise a desired agent. The desired agent can be, for example, any colorant (pigment, dye, etc.), fiber or any additional film forming agent known in the art.

Acceptable colorants include pigments, dyes, such as liposoluble dyes, nacreous pigments, and pearling agents.

Representative liposoluble dyes which may be used according to the present invention include Sudan Red, DC Red 17, DC Green 6, ß-carotene, soybean oil, Sudan Brown, DC Yellow 11, DC Violet 2, DC Orange 5, annatto, and quinoline yellow.

Representative nacreous pigments include white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium,

Suitable fibers include, but are not limited to, fibers which enable improvement of the lengthening effect. “Fiber” should be understood to mean an object of length L and diameter D such that L is much greater than D, D being the diameter of the circle in which the cross-section of the fibre is inscribed. In particular, the L/D ratio (or form factor) is selected in the band ranging from 3.5 to 2500, in particular from 5 to 500, and more particularly from 5 to 150. The fibers utilisable in the composition of the invention can be fibers of synthetic or natural origin, mineral or organic. They can be short or long, unitary or structured, for example, braided, hollow or full. They can be of any shape and in particular of circular or polygonal cross-section (square, hexagonal or octagonal) depending on the specific application envisaged. In particular, their ends are blunted and/or polished to avoid injury. They can be rigid or non-rigid fibers. They can be of synthetic or natural origin, mineral or organic. They can be surface treated or not, coated or not, and colored or not.

If present, the desired agent (colorant; pigment) is preferably present in the compositions of the present invention in an amount of from 0.1% to 30% by weight, preferably in an amount of from 0.5% to 25%, preferably in an amount of from 1% to 20% by weight, such as 5% to about 15% by weight, based on the total weight of the composition.

According to certain notable embodiments, the compositions of the present invention are W/O emulsions and include water, oil, wax, at least one surfactant, at least one amine salt of an acrylic polymer, at least one organosiloxane film-former that is selected from a silicone resin and a random acrylic/silicone graft copolymer, at least one colorant, and optionally at least one silicate clay. According to certain other embodiments at least one of the following concentration by weight limitations are also characteristic of the compositions: about 15% to about 30% water, about 15% to about 30% oil, about 10% to about 25% by weight of wax, about 2% to about 8% of at least one surfactant, about 3% to about 12% of at least one amine salt of an acrylic polymer, about 5% to 25% by weight at least one organosiloxane film-former (such as one without styrene groups) that is selected from a silicone resin and a random acrylic/silicone graft copolymer, about 5% to about 15% at least one colorant, and optionally about 1% to about 5% of at least one silicate clay and about 1% to about 5% by weight silicone gum. These compositions may be substantially free or free of acrylic polymers that include styrene groups.

According to other preferred embodiments, methods of treating, caring for and/or enhancing the appearance of hair, eyebrows or eyelashes by applying compositions of the present invention to the hair, eyebrows or eyelashes in an amount sufficient to treat, care for and/or enhance the appearance of the hair, eyebrows or eyelashes are provided. In accordance with these preceding preferred embodiments, the compositions of the present invention are applied topically to the desired area of the hair, eyebrows or eyelashes in an amount sufficient to treat, care for and/or enhance the appearance of the hair, eyebrows or eyelashes. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, improved removal and/or improved sebum resistance are provided. Preferably, the composition is a composition for eyelashes. Most preferably, the composition is a mascara composition further comprising at least one colorant.

According to a preferred embodiment of the present invention, methods of making a water-in-oil (w/o) emulsion for hair, eyelashes and/or eyebrows comprising combining at least one low HLB surfactant, at least one silicone gum, at least one amine salt of an acrylic polymer, at least one organosiloxane film-former that is selected from a silicone resin and a random acrylic/silicone graft copolymer, and optionally at least one wax, to form the emulsion are provided. Preferably, the composition is a composition for eyelashes. Most preferably, the composition is a mascara composition further comprising at least one colorant. Most preferably, the composition comprises 26% or less by weight of wax, if present.

According to a preferred embodiment of the present invention, compositions having improved cosmetic properties such as, for example, increased removability from hair, eyebrows or eyelashes to which they have been applied are provided. Preferably, the composition is a composition for eyelashes. Most preferably, the composition is a mascara composition further comprising at least one colorant.

According to a preferred embodiment of the present invention, methods of improving the removal properties (from hair, eyebrows and/or eyelashes) of a water-in-oil (w/o) emulsion composition comprising at least one organosiloxane film-former that is selected from a silicone resin and a random acrylic/silicone graft copolymer, and optionally at least one wax, by adding at least one amine salt of an acrylic polymer in an amount sufficient to add from about 2% to about 6% by weight with respect to the total weight of the composition to the emulsion composition, resulting in increased removal properties of the emulsion composition, are provided. Preferably, the composition is a composition for eyelashes. Most preferably, the composition is a mascara composition further comprising at least one colorant.

According to a preferred embodiment of the present invention, methods of making a water-in-oil (w/o) emulsion composition for hair, eyelashes and/or eyebrows comprising combining at least one amine salt of an acrylic polymer an amount sufficient to add from about 2% to about 6% by weight with respect to the total weight of the composition and at least one organosiloxane film-former that is selected from a silicone resin and a random acrylic/silicone graft copolymer, and optionally at least one wax, to form the emulsion composition are provided. Preferably, the composition is a composition for eyelashes. Most preferably, the composition is a mascara composition further comprising at least one colorant.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Mascara Composition

An example inventive composition is shown in Table 4 below.

TABLE 4

| | | Total %100.00 |
|---|---|---|
| Phase | Chemical Name | % wt/wt |
| A | Low HLB Surfactants | 5 |
| A | Waxes | 15 |
| A | Silicone Gum | 2 |
| A | Oils | 25 |
| A | Preservatives | 1 |
| A | Colorants -- Iron Oxides | 8 |
| A | Polyol | 2 |
| A | Trimethylsiloxsilicate | 8 |
| A | Clays | 3 |
| B | Water | 27 |
| B | Styrene/acrylates/ammonium | 4 |
| | methacrylate copolymer | |

Sample Procedure for Preparing Compositions of the Present Invention:

In metal container A, All phase A materials are added and heated until 90° C.

When metal container A was at 90° C., homogenized for 30 minutes at 2000 RPM.

After the mixture was uniform and cooled to 45° C., added B subsequently, and mixed for 10 mins.

Batch Cooled to 30° C.

### Example 2

Sebum Resistance Testing and Removal Testing Artificial sebum protocol: on fake lashes, apply 3×10 strokes of composition with 2 min dry time in between. Lashes then dipped in solvent 1h after application of composition. Lashes then left in the solvent for 1h and then placed in the spinning apparatus where the lashes rub against a sheet of drawing paper. 3 samples of each formula were assessed on one sheet. The sheets are then scanned as black and white images, and uploaded to an imaging analysis software, ImageJ, an image processing program developed at the National Institutes of Health and the Laboratory for Optical and Computational Instrumentation. The black smudges are counted as % black pixel area, and the higher the % black pixel area, the worse the smudging.

Removal method protocol: on fake lashes, apply 3×10 strokes of composition with 2 min dry time in between. Removal carried out 1 h after application. 1.5 g of GARNIER (pink) MICELLAR CLEANSING WATER added to cotton pad. Lashes softly pinched for 10 sec. with the soaked pad and then the cotton pad is removed by softly pulling under a neutral grip. The cotton pads are then counted and evaluated for the amount of product removed per pad. This is compared to a standard formula (including the same polymers as Sample 1), and the less amount of pads required for removal, the easier the removal is considered. Ratings are 1-5, with 1-2 being worse than the standard, 3 being the same as the standard, and 4-5 being better than the standard.

Compositions falling with the ranges set forth in the following Table 5 were prepared and tested for both ease of removal and wear properties according to the protocol described below.

TABLE 5

| Phase | Chemical Name | % wt/wt |
|---|---|---|
| A | Isododecane | 42 |
| A | Low HLB Surfactants | 4 |
| A | Silicone Gum | 3 |
| A | Colorants -- Iron Oxides | 8 |
| A | Organosiloxane film-former or other | 8 |
| | oil-phase polymer | |
| A | Clays | 3 |
| B | Water | 29 |
| B | Ammonium salt of an acrylic polymer | 4 |
| | or other water-phase polymer | |
| | | Total %100.00* |

*Apparent Addition Error due to rounding

The specific following pairs of oil phase/water phase polymers were tested, as shown in Table 6, with results for Sebum Remaining and Ease of Removal reported:

TABLE 6

| | | | Sebum | Ease of |
|---|---|---|---|---|
| SAMPLE | Oil-phase Polymer | Water-phase polymer | Remaining | Removal |
| 1 | Trimethylsiloxysilicate | Styrene/Acrylates/Ammonium | 0.1 | 3 |
| | | methacrylate copolymer | | |
| 2 | Trimethylsiloxysilicate | Ammonium acrylates copolymer | 0.359 | 4 |
| 3 | Polypropylsilsesquioxane | Styrene/Acrylates/Ammonium | 0.386 | 5 |
| | | methacrylate copolymer | | |
| 4 | Random acrylic/silicone | Styrene/Acrylates/Ammonium | 0.144 | 5 |
| | graft copolymer | methacrylate copolymer | | |
| 5 | Trimethylsiloxysilicate | Acrylates copolymer | 0.252 | 2 |
| 6 | Trimethylsiloxysilicate | Polyurethane-35 | 0.147 | 1 |
| 7 | Trimethylsiloxysilicate | Acrylates/ethylhexyl acrylate | 0.027 | 2 |
| | | copolymer and Acrylates/dimethyl | | |
| | | aminoethyl methacrylate copolymer | | |
| 8 | VP/Hexadecene Copolymer | Styrene/Acrylates/Ammonium | 6.155 | 5 |
| | | methacrylate copolymer | | |
| 9 | VP/Eicosene Copolymer | Styrene/Acrylates/Ammonium | 6.1 | 5 |
| | | methacrylate copolymer | | |
| 10 | Polyvinyl laurate | Styrene/Acrylates/Ammonium | 2.957 | 5 |
| | | methacrylate copolymer | | |
| 11 | Block copolymer silicone | Styrene/Acrylates/Ammonium | 3.867 | 5 |
| | acrylate | methacrylate copolymer | | |

As shown in Table 6, for cases in which an amine salt of an acrylic polymer was chosen as the water phase polymer and an organosiloxane film-former that is selected from a silicone resin and a random acrylic/silicone graft copolymer was chosen as the oil-phase polymer (Samples 1, 2, 3 and 4), one is able to achieve both good sebum resistance (less than 1.0) and simultaneously have good ease of removal (3 or higher).

What is claimed is:

1. A water-in-oil emulsion composition comprising (a) water, (b) at least one oil, (c) at least one surfactant, (d) at least one amine salt of an acrylic polymer, and (e) at least one organosiloxane film-former that is selected from a silicone resin and a random acrylic/silicone graft copolymer.

2. The composition of claim 1, wherein the amine salt of an acrylic polymer is free of styrene groups.

3. The composition of claim 1, further comprising at least one silicone gum.

4. The composition of claim 1, wherein the composition comprises from about 2% to about 6% by weight with respect to the total weight of the composition of the at least one amine salt of an acrylic polymer.

5. The composition of claim 1, wherein the at least one surfactant is at least one low HLB surfactant.

6. The composition of claim 1, wherein the at least one organosiloxane film-former is a trimethylsiloxysilicate resin.

7. The composition of claim 1, wherein the amine salt of an acrylic polymer is an ammonium salt of an acrylic polymer.

8. The composition of claim 1, further comprising at least one wax in an amount from about 10% to about 25% by weight of the total weight of the composition.

9. The composition of claim 1, further comprising at least one colorant.

10. The composition of claim 1, further comprising at least one silicate clay.

11. The composition of claim 1, wherein the organosiloxane film-former that is selected from a silicone resin and a random acrylic/silicone graft copolymer is present in an amount from about 1% to about 10% by weight of the total weight of the composition and/or the weight ratio of organosiloxane film-former that is selected from a silicone resin and a random acrylic/silicone graft copolymer to amine salt of an acrylic polymer is present in a range from 1:1 to about 5:1, preferably from 2:1 to about 4:1.

12. The composition of claim 1, in the form of a mascara.

13. The composition of claim 1, wherein the composition is substantially free of acrylic polymers that comprise styrene groups.

14. The composition of claim 10 wherein the silicate clay is hydrophobic.

15. The composition of claim 1 wherein the composition comprises at least 1% by weight of one or more waxes having a melting point of greater than 65° C.

16. A method of making up eyelashes comprising applying the composition according to any preceding claim, of claim 1 to eyelashes.
